(19) United States
(12) Patent Application Publication
(10) Pub. No.: US 2024/0285485 A1
(43) Pub. Date: Aug. 29, 2024

(54) WATER-IN-OIL TYPE SOLID EMULSIFIED COSMETIC PREPARATION

(71) Applicant: TOSHIKI PIGMENT CO., LTD., Tokyo (JP)

(72) Inventor: Kazuyoshi TSUBATA, Tokyo (JP)

(21) Appl. No.: 18/586,718

(22) Filed: Feb. 26, 2024

(30) Foreign Application Priority Data
Feb. 27, 2023 (JP) 2023-028028
Feb. 13, 2024 (JP) 2024-019701

(51) Int. Cl. A61K 8/06; A61K 8/31; A61K 8/365; A61K 8/37; A61K 8/46; A61K 8/92; A61Q 1/06; A61Q 1/08; A61Q 1/10

(52) U.S. Cl. CPC A61K 8/064; A61K 8/31; A61K 8/365; A61K 8/37; A61K 8/46; A61K 8/922; A61K 8/927; A61Q 1/06; A61Q 1/08; A61Q 1/10; A61K 2800/34

(57) ABSTRACT

Provided is a water-in-oil type solid emulsified cosmetic preparation with good feelings from use. The water-in-oil type solid emulsified cosmetic preparation, contains, and being substantially free of silicone compounds, a compound which is pasty at 25° C.; a compound selected from PEG-30 dipolyhydroxystearate, PEG-5 hydrogenated castor oil, PEG-10 hydrogenated castor oil, PEG-20 hydrogenated castor oil, PEG-30 hydrogenated castor oil, PEG-40 hydrogenated castor oil, PEG-60 hydrogenated castor oil and a combination of these; a solid oil component having a melting point of 65° C. or more; a low-viscosity liquid oil component having a viscosity of 40 mPa·s or less at 25° C.; and water.

### FIELD OF THE INVENTION

The present invention relates to water-in-oil type solid emulsified cosmetic preparations, for example, water-in-oil type solid emulsified cosmetic preparations such as foundations, eye shadows, eye liners, mascaras, concealers, lipsticks, balms and others.

### BACKGROUND OF THE INVENTION

Conventionally, water-in-oil type emulsified cosmetic preparations formulated with volatile silicone oil agents, silicone-based emulsifiers or the like have been developed to reduce feelings from use when oily cosmetic preparations are applied (sticky feeling, heavy spreadability) or feelings after the application (sticky feeling, filmy feeling).

JP-A 2010-132619 discloses a water-in-oil type emulsified make-up cosmetic preparation containing glyceryl monoisostearate, decamethylcyclopentasiloxane, which is a liquid oil component, and a solid oil component.

JP-A 2019-108286 discloses a water-in-oil type emulsified composition containing PEG-30 dipolyhydroxystearate, (phytosteryl/isostearyl/cetyl/stearyl/behenyl) dimer dilinoleate as a semisolid ester oil, diglycerin and water.

JP-A 2022-53332 describes a composition for external use on skin containing an (ethylene/propylene) copolymer and PEG-30 dipolyhydroxystearate.

The cosmetic preparation and compositions described in the above prior art patent literatures can be formulated with silicone compounds to obtain good spreadability without stickiness, and other feelings from use or to improve dispersibility of powder components. However, as silicone compounds do not have biodegradability, their accumulation due to a long period of use has been a problem in recent years.

WO-A 2022-092128 describes a water-in-oil type emulsified sunscreen cosmetic preparation which is free of silicones.

### SUMMARY OF THE INVENTION

### Problems to be Solved by the Invention

A problem to be solved by the present invention is to provide a water-in-oil type solid emulsified cosmetic preparation which is substantially free of silicones and reduces a sticky feeling or heavy spreadability when it is applied or a sticky feeling or filmy feeling after it is applied. Further, another problem to be solved by the present invention is to provide a water-in-oil type solid emulsified cosmetic preparation which is also excellent in handleability in a production process or stability of products after production.

### Solution to the Problems

The present invention provides a water-in-oil type solid emulsified cosmetic preparation, containing the following components, and substantially free of silicone compounds,
(A) a compound which is pasty at 25° C. (hereinafter referred to as “component (A)”),
(B) a compound selected from PEG-30 dipolyhydroxystearate, PEG-5 hydrogenated castor oil, PEG-10 hydrogenated castor oil, PEG-20 hydrogenated castor oil, PEG-30 hydrogenated castor oil, PEG-40 hydrogenated castor oil, PEG-60 hydrogenated castor oil and a combination of these (hereinafter referred to as “component (B)”),
(C) a solid oil component having a melting point of 65° C. or more (hereinafter referred to as “component (C)”),
(D) a low-viscosity liquid oil component having a viscosity of 40 mPa·s or less at 25° C. (hereinafter referred to as “component (D)”), and
(E) water (hereinafter referred to as “component (E)”).

### Advantageous Effects of the Invention

The water-in-oil type solid emulsified cosmetic preparation of the present invention is substantially free of silicones, and reduces a sticky feeling or heavy spreadability when it is applied and further a sticky feeling or filmy feeling after it is applied, thus being able to give good feelings from use. Further, it has good handleability in a production process, and is also excellent in stability of products after production.

### EMBODIMENTS OF THE INVENTION

### <Water-In-Oil Type Solid Emulsified Cosmetic Preparation>

The water-in-oil type solid emulsified cosmetic preparation of the present invention (hereinafter referred to as “the cosmetic preparation of the present invention”) can be made of an oil phase containing components (A), (B), (C) and (D), a water phase containing component (E), and optionally, a powder phase containing (F) cellulose (hereinafter referred to as “component (F)”), or the like as necessary, and other cosmetic preparation components.

The compound of component (A) used in the cosmetic preparation of the present invention is pasty at 25° C. The compound which is pasty at 25° C. has a viscosity of preferably more than 1,000 mPa·s and 100,000 mPa·s or less and more preferably more than 5,000 mPa·s and 50,000 mPa·s or less at 25° C. Here, the viscosity is a viscosity measured by a viscoelasticity measurement device (rheometer) at 25° C. and a shearing rate of 10 (1/s).

As the compound of component (A), one or two or more selected from hydrogenated castor oil isostearate, dipentaerythrityl hexahydroxystearate, dipentaerythrityl hexa (hydroxystearate/stearate/rosinate), dipentaerythrityl tri-polyhydroxystearate, bis-diglyceryl polyacyladipate-2, phytosteryl macadamiate, (phytosteryl/isostearyl/cetyl/stearyl/behenyl) dimer dilinoleate, di(phytosteryl/octyldodecyl) lauroyl glutamate, di(octyldodecyl/phytosteryl/behenyl) lauroyl glutamate and bis(behenyl/isostearyl/phytosteryl) dimer dilinoleyl dimer dilinoleate can be used.

As component (A), bis-diglyceryl polyacyladipate-2 and hydrogenated castor oil isostearate are preferable and bis-diglyceryl polyacyladipate-2 is preferable from the viewpoints of feelings from use and temporal stability.

Further, in the cosmetic preparation of the present invention, bis-diglyceryl polyacyladipate-2 and hydrogenated castor oil isostearate are preferably used in combination as component (A) from the viewpoint of dispersibility of the powder phase or the like.

A content of component (A) in a total amount of the cosmetic preparation of the present invention is preferably 4 to 15 mass % and more preferably 6 to 13 mass %.

The compound of component (B) used in the cosmetic preparation of the present invention is a compound selected from PEG-30 dipolyhydroxystearate, PEG-5 hydrogenated castor oil, PEG-10 hydrogenated castor oil, PEG-20 hydrogenated castor oil, PEG-30 hydrogenated castor oil, PEG-40 hydrogenated castor oil, PEG-60 hydrogenated castor oil and a combination of two or more of these. When a combination of these compounds is used, it can be used as a mixture during production of cosmetic preparations.

Component (B) is more preferably PEG-20 hydrogenated castor oil from the viewpoint of feelings from use.

Component (B) is preferably a combination of PEG-30 dipolyhydroxystearate (hereinafter referred to as component (B1)) and one or more selected from PEG-5 hydrogenated castor oil, PEG-10 hydrogenated castor oil, PEG-20 hydrogenated castor oil, PEG-30 hydrogenated castor oil, PEG-40 hydrogenated castor oil and PEG-60 hydrogenated castor oil (hereinafter referred to as component (B2)) from the viewpoint of handleability in a production process or temporal stability and feelings from use of products after production.

When component (B) is a combination of components (B1) and (B2), a mass ratio between them, (B1)/(B2), preferably falls within the range of 1 to 5 and more preferably falls within the range of 1 to 3.

A content of component (B) in a total amount of the cosmetic preparation of the present invention is preferably 0.5 to 10 masse, more preferably 1 to 8 mass % and further preferably 1.5 to 6 mass %.

Component (C) used in the cosmetic preparation of the present invention is a solid oil having a melting point of 65° C. or more.

As component (C), a plant-derived wax ester and a hydrocarbon-based wax are preferable, and specifically, one or two or more selected from solid waxes such as sunflower seed wax, synthetic waxes, beeswax, candelilla wax, carnauba wax, rice bran wax, microcrystalline waxes, polyethylene, (ethylene/propylene) copolymers, solid paraffin, sumac wax, ozokerite, ceresin, hydrogenated hardened oils and others can be used.

Component (C) is preferably one or two or more selected from sunflower seed wax, synthetic waxes, beeswax, candelilla wax, carnauba wax, rice bran wax, microcrystalline waxes and (ethylene/propylene) copolymers from the viewpoints of feelings from use and temporal stability.

The synthetic waxes refer to “hydrocarbon-based waxes obtained by the Fischer-Tropsch process or ethylene polymerization processes,” as defined in “List of Labeling Names of Cosmetic Ingredients” prepared by Japan Cosmetic Industry Association.

A content of component (C) in a total amount of the cosmetic preparation of the present invention is preferably 5 to 20 mass % and more preferably 8 to 15 mass %.

Component (D) used in the cosmetic preparation of the present invention is a low-viscosity liquid oil having a viscosity of 40 mPa·s or less at 25° C., and preferably a low-viscosity liquid oil having a viscosity of 15 mPa·s or less. The viscosity may be obtained, for example, by measuring a kinematic viscosity and then converting it thereto, and a method for measuring the kinematic viscosity is not particularly limited as long as it is a general one, but measurement methods such as ISO 23581:2020 “Petroleum products and related products-Determination of kinematic viscosity-Method by Stabinger type viscometer,” ISO 12185:1996 “Crude petroleum and petroleum products-Determination of density-Oscillating U-tube method” and others are suitable.

As component (D), one or two or more selected from hexyl laurate, isoamyl laurate, isopropyl myristate, isocetyl myristate, octyldodecyl myristate, isopropyl palmitate, ethylhexyl palmitate, isocetyl stearate, ethyl isostearate, isopropyl isostearate, hexyldecyl isostearate, isostearyl isostearate, ethylhexyl isononanoate, isobutyl isostearate, cetyl ethylhexanoate, isostearyl neopentanoate, isononyl isononanoate, isodecyl isononanoate, isotridecyl isononanoate, tricyclodecanemethyl isononanoate, jojoba oil, neopentyl glycol diethylhexanoate, neopentyl glycol dicaprate, neopentyl glycol diisononanoate, isostearyl neopentanoate, bis-ethoxydiglycol succinate, diethoxyethyl succinate, diethylhexyl succinate, glyceryl tri (caprylate/caprate), triethylhexanoin, mineral oil, squalane, dodecane, isododecane, tetradecane, hexadecane and isohexadecane can be used.

Component (D) is preferably one or two or more selected from ethylhexyl palmitate, triethylhexanoin, glyceryl tri (caprylate/caprate), dodecane, isododecane and isohexadecane from the viewpoints of a feeling from use (light spreadability) and temporal stability (hardness).

Further, component (D) is more preferably a combination of ethylhexyl palmitate and triethylhexanoin from the viewpoint of a feeling from use (light spreadability).

Further, component (D) is more preferably a combination of triethylhexanoin and one or more selected from dodecane, isododecane and isohexadecane from the viewpoints of a feeling from use (light spreadability) and temporal stability (hardness).

A content of component (D) in a total amount of the cosmetic preparation of the present invention may be 20 to 50 mass, and is preferably 30 to 45 mass % and more preferably 32 to 42 mass %.

In the cosmetic preparation of the present invention, a total content of components (A), (C) and (D) as a combining amount of oil components excluding component (B) is preferably 23 to 70 mass % and more preferably 40 to 60 mass %. If the total content falls within the above range, emulsification can be performed stably, and further sufficient feelings from use (moistness, light spreadability) can be obtained.

Water of component (E) used in the cosmetic preparation of the present invention is preferably purified water from a hygiene viewpoint. A content of component (E) in a total amount of the cosmetic preparation of the present invention is preferably 10 to 70 mass % and more preferably 20 to 50 mass %.

Further, a mass ratio of a content of component (B) to a content of component (E) in the above cosmetic preparation, (B)/(E), may fall within the range of 0.01 to 0.5, and preferably falls within the range of 0.02 to 0.4 and more preferably falls within the range of 0.07 to 0.3. If the mass ratio falls within the above range, good handleability in a production process can be attained, and excellent feelings from use and temporal stability of products are achieved.

The cosmetic preparation of the present invention can further optionally contain cellulose of component (F).

Component (F) is preferably a spherical cellulose, and an average particle size of the spherical cellulose is preferably 1 to 150 μm and more preferably 5 to 50 μm.

As the spherical cellulose, cellulose itself can be used, and cellulose derivatives such as acetylcellulose and others can be used. For example, “GE-800” (manufactured by TOSHIKI PIGMENT CO., LTD.) can be used.

Further, a hydrophobized spherical cellulose which has been subjected to a surface treatment using a hydrophobizing agent as necessary can be used. Examples of the hydrophobizing agent used as necessary include metal soaps such as zinc stearate, magnesium stearate and others, amino acids such as disodium stearoyl glutamate and others, or the like. Silicone-treated spherical celluloses which have been subjected to surface treatments with silicone compounds are not included in component (F).

A content of component (F) in a total amount of the cosmetic preparation of the present invention is preferably 1 to 20 mass % and more preferably 1.5 to 15 mass %.

In the cosmetic preparation of the present invention, a total of components (A), (B), (C), (D) and (E), and component (F) as necessary, and components of the balance including the other cosmetic preparation components is 100 mass %.

The “components of the balance including the other cosmetic preparation components” are components not involved in the expression of the effects of the invention, but for molding water-in-oil type solid emulsified cosmetic preparations or expressing functions as cosmetic preparations (for example, a coloring effect of a colorant).

In the cosmetic preparation of the present invention, a mass ratio of a content of component (A) to a content of component (B), (A)/(B), preferably falls within the range of 0.6 to 10, more preferably falls within the range of 1.2 to 10 and further preferably falls within the range of 1.5 to 8.

In the cosmetic preparation of the present invention, a mass ratio of a content of component (B) in the above cosmetic preparation to a total content of components (A), (B), (C) and (D) in the above cosmetic preparation, (B)/[(A)+(B)+(C)+(D)], is preferably 0.016 to 0.2 and more preferably 0.02 to 0.15.

### <Other Components>

The cosmetic preparation of the present invention may contain the following cosmetic preparation components as the other cosmetic preparation components:
a higher alcohol such as behenyl alcohol, octyldodecanol or the like;
a polysaccharide fatty acid ester such as dextrin palmitate or the like;
a polyglyceryl fatty acid ester such as polyglyceryl-10 stearate or the like;
an antioxidant such as d-δ-tocopherol or the like;
rosin pentaerythritol ester, neopentyl glycol diisooctanoate, glyceryl triisooctanoate, propylene glycol monolaurate, polyglyceryl-10 pentaisostearate or the like;
as a solvent, ethanol, isopropanol, a polyhydric alcohol, a water-soluble polymer, bentonite, polyoxyethylene sorbitan monostearate, sorbitan sesquiisostearate, ethyl acetate, butyl acetate, butanol;
as a moisturizer, glycerin, propylene glycol, sorbitol, polyethylene glycol, dipropylene glycol, 1,3-butylene glycol, pentylene glycol, diglycerin, mannitol, POE methyl glycoside, a biopolymer, sucrose or the like;
an emollient component such as cetyl ethylhexanoate or the like;
quince seed, pectin, a cellulose derivative, xanthan gum, sodium alginate, soageena, carboxy vinyl polymer;
sorbitan monooleate, glyceryl triisooctanoate, octyl methoxycinnamate, POE sorbitan monooleate, isostearic acid, stearic acid, acetyl tributyl citrate, trimethylpentanediyl dibenzoate, stearalkonium bentonite;
a thickener, a preservative, an emulsifier, a stabilizer, a plasticizer;
an anionic surfactant, a cationic surfactant, a nonionic surfactant, an amphoteric surfactant, a protein-based surfactant;
a medicinal component, vitamin C dipalmitate, vitamins, an amino acid, a whitening agent, a bactericide;
an extender pigment such as talc, kaolin, mica, sericite, alumina, silica, glass flakes, calcium carbonate, magnesium carbonate, silicic anhydride, barium sulfate or the like, a pearlescent pigment;
a pH adjuster, potassium hydroxide, sodium hydroxide, triethanolamine, magnesium stearate, zinc stearate, a metal ion sequestering agent, citric acid, sodium citrate, lactic acid, sodium lactate;
a UV absorber, an antioxidant, a fragrance, a preservative, a chelating agent, an antioxidant, a dispersant, an anti-browning agent, a buffer, an anti-precipitation component, an organically modified clay mineral, a usability modifier;
as a powder phase, a metal oxide such as bengara (iron oxide), yellow iron oxide, black iron oxide, titanium oxide, chromium oxide or the like;
(HDI/trimethylol hexyllactone) crosspolymer; and
photoluminescent powders such as bismuth oxychloride, mica titanium, iron oxide coated mica, iron oxide mica titanium, organically pigmented mica titanium, aluminum powder, a pearlescent pigment or the like.

Note that, in the cosmetic preparation of the present invention, silicone-containing materials are not used in raw materials for producing the above-listed other components, and silicone-treated powder components are not used in the above-listed other powder components, either.

The cosmetic preparation of the present invention is substantially free of silicone compounds. Being “substantially free of silicone compounds” means that silicone compounds are not intentionally added to the cosmetic preparation of the present invention. However, the possibility that minute amounts of silicone compounds are mixed into cosmetic preparation components including the above components (A) to (F) and optional components in production steps thereof cannot be ruled out, and the possibility that minute amounts of silicone compounds are in some cases contained in each component as formulation components cannot be ruled out, and thus, the cosmetic preparation of the present invention also encompasses those containing preferably 0.01 mass % or less and more preferably 0.001 mass % or less of silicone compounds (as a silicon content).

Publicly-known measurement methods can be used for measuring the silicone compounds content. For example, the silicon content of the silicone compounds can be measured on the basis of descriptions in “Determination of Dimethylpolysiloxane in Foods by Atomic Absorption Spectrophotometry” (Motohiro NISHIJIMA et al., Food Hygiene and Safety Science, Vol. 16, No. 2, pp. 110-115) or “Analysis of Silicone Oil in Sludge by Atomic Absorption Spectrophotometry” (Yuichi TSUCHIYA et al., Japan Analyst, Vol. 27 (1978), No. 6, pp. 343-346).

The water-in-oil type solid emulsified cosmetic preparation of the present invention can be produced, for example, by a production method including the following first to fourth steps,
first step: a step of melt-kneading the oil phase containing components (A), (B), (C) and (D),
second step: a step of as necessary adding the powder phase containing optional component (F) to the kneaded product obtained in the first step, and dispersing or kneading it therein,
third step: a step of adding heated water of component (E) to the disperse product or kneaded product obtained in the second step to perform emulsification, and
fourth step: a step of as necessary defoaming the emulsified product obtained in the third step, and then filling or molding it in a container.

In the first step, oil phase components including components (A), (B), (C) and (D) can be melted by heating them to 80 to 100° C. and using a paddle mixer or the like to obtain the kneaded product.

The above oil phase may contain optional cosmetic preparation components (oil phase) in liquid form, gel form or solid form in addition to components (A), (B), (C) and (D).

The second step is performed while the kneaded product obtained in the first step is heated, for example, at 80 to 100° C. The powder phase containing component (F) and optional powdery cosmetic preparation components is as necessary added to the above kneaded product in a molten state and mixed uniformly using a three roll mill or the like to obtain the disperse product or kneaded product in a molten state in which the powder phase is dispersed in the oil phase.

In the third step, water of component (E) which is heated, for example, to 80 to 100° C. is added to the disperse product or kneaded product obtained in the second step and dispersed with a homogenizer or the like to obtain a water-in-oil type solid emulsified cosmetic preparation composition. In this step, optional components may be added.

In the fourth step, the composition obtained in the third step is heated, for example, to 80 to 100° C., defoamed while stirred using a vacuum defoamer, and then filled or molded in the container to obtain the water-in-oil type solid emulsified cosmetic preparation of the present invention.

The cosmetic preparation of the present invention is substantially free of silicone compounds, and thus does not cause phase separation even if it is cooled during the above production steps, for example, during or immediately after the first step, during or immediately after the second step, or during or immediately after the third step. Particularly, even if it is cooled during or immediately after the third step in which water of component (E) is added, re-emulsification is easy to perform.

In the cosmetic preparation of the present invention, a content of the powder phase made of component (F) and other components such as a permitted colorant, an extender pigment and others depends on the types of cosmetic preparations, but is preferably 1 to 30 mass % and more preferably 3 to 20 mass % in the above cosmetic preparation in the above production method. The balance is a content of the oil phase containing components (A), (B), (C) and (D) and the water phase containing component (E).

### EXAMPLES

### <Used Components>

### Component (A)

Hydrogenated castor oil isostearate: product name “CASTORIDE MIS-P” (pasty at 25° C.) manufactured by National Mimatsu Co., Ltd.
Bis-diglyceryl polyacyladipate-2: product name “SOFTISAN 649” (pasty at 25° C.) manufactured by IOI Oleo GmbH

### Component (B)

PEG-30 dipolyhydroxystearate: product name “CITHROL DPHS” manufactured by Croda International Plc
PEG-20 hydrogenated castor oil: product name “NIKKOL HCO-20” manufactured by Nikko Chemicals Co., Ltd.

### Component (C)

Synthetic wax: product name “PERFORMA SW-88 Synthetic Wax” manufactured by NuCera Solutions LLC
Beeswax: product name “BEES WAX” manufactured by Miki Chemical Industry Co., Ltd.
Sunflower seed wax: product name “Sunflower Wax” manufactured by YOKOZEKI OIL & FAT INDUSTRIES CO., LTD.

### Component (D)

Triethylhexanoin: product name “Trifat S-308” manufactured by NIPPON SURFACTANT INDUSTRIES CO., LTD.
Ethylhexyl palmitate: product name “IOP” manufactured by Nikko Chemicals Co., Ltd.
Dodecane: product name “PARAFOL 12-97” manufactured by SASOL Germany GmbH

### Component (F)

Cellulose (spherical): “GE-800” manufactured by TOSHIKI PIGMENT CO., LTD.

### Comparative Component (B′)

PEG-20 glyceryl triisostearate: product name “NIKKOL TGI-20” manufactured by NIPPON SURFACTANT INDUSTRIES CO., LTD.
PEG-100 hydrogenated castor oil: product name “NIKKOL HCO-100” manufactured by Nikko Chemicals Co., Ltd.

The water-in-oil type solid emulsified cosmetic preparations of the examples and comparative examples were prepared by the method below.

The oil phases containing components (A) to (D) shown in Tables 1 and 2 were each melt-kneaded at 90 to 100° C. using a paddle mixer (first step).

A powder phase containing component (F) was added to the melted oil phase and kneaded three times with a three roll mill to obtain a kneaded product (second step). After heated to a temperature in the neighborhood of 90° C., the kneaded product was mixed with component (E) at 90 to 100° C. and further mixed with optional components shown in the tables, thus obtaining the water-in-oil type solid emulsified cosmetic preparation compositions of the examples and comparative examples (third step).

The obtained cosmetic preparation compositions were each defoamed while stirred using a vacuum defoamer, and then filled into a predetermined container to obtain the water-in-oil type solid emulsified cosmetic preparations. The items below were evaluated using the above cosmetic preparations, and the results are all together shown in Tables 1 and 2.

### <Method for Evaluating Cosmetic Preparation>

### Moldability

The cosmetic preparation compositions obtained in the examples and comparative examples were each melted at about 90° C., filled into a predetermined mold for rod-shaped cosmetic preparations (inner diameter 12 mm×length 44 mm, 4.0 g in capacity), and left to stand at −20° C. for 5 minutes to be solidified. After the solidified rod-shaped cosmetic preparation was filled into a predetermined container and left to stand at a room temperature for 6 hours, an appearance of the cosmetic preparation was visually observed, and moldability was evaluated in accordance with the following criteria.
A: the cosmetic preparation could be easily removed from the mold and could be filled into the predetermined container, and it could be confirmed that the surface thereof was uniform
B: the cosmetic preparation could be removed from the mold and could be filled into the predetermined container, but scratches caused by rubbing during the removal from the mold could be observed on the surface thereof
C: the cosmetic preparation was incompletely solidified and could not be removed from the mold

### Dispersion Stability and Emulsification Stability Under Remelting

The water-in-oil type solid emulsified cosmetic preparation composition after the third step was cooled by leaving it to stand at a room temperature (about 25° C.) for 6 hours or more. After it was confirmed that the cosmetic preparation composition had been completely solidified, the solidified cosmetic preparation composition was turned into a remelted state at about 90° C. while stirred with a propeller mixer (at 30 rpm), and evaluated in accordance with the following criteria.
A: there were neither precipitations nor flocculants, and dispersion was homogeneous
B: some precipitations or flocculants were observed, and dispersion was slightly heterogenous
C: precipitations were observed

### Usability (Light Spreadability)

Five female cosmetic evaluation panelists applied each of the cosmetic preparations of the examples and comparative examples to their skin in a routine manner, and evaluated it in accordance with the following criteria. The most-given evaluation was employed as the evaluation result.
A: the cosmetic preparation spread lightly when applied, and gave a good feeling
B: the cosmetic preparation spread somewhat lightly when applied, and gave a somewhat good feeling
C: the cosmetic preparation spread heavily when applied, and did not give a good feeling

### Usability (Moistness)

Five female cosmetic evaluation panelists applied each of the cosmetic preparations of the examples and comparative examples to their skin in a routine manner, and evaluated usability (moistness) in accordance with the following criteria.
A: of the five persons, all of them evaluated the cosmetic preparation as moist
B: of the five persons, 2 to 4 persons evaluated the cosmetic preparation as moist
C: of the five persons, one or less person evaluated the cosmetic preparation as moist

### Temporal Stability of Cosmetic Preparation (Appearance)

The cosmetic preparation compositions obtained in the examples and comparative examples were each melted at about 90° C., filled into a predetermined mold for rod-shaped cosmetic preparations (inner diameter 12 mm×length 44 mm, 4.0 g in capacity), and left to stand at −20° C. for 5 minutes to be solidified. The solidified rod-shaped cosmetic preparation was filled into a predetermined container, and left to stand at a room temperature for 6 hours or more. The cosmetic preparation was placed in a cycle testing thermostatic chamber, and a cycle test was conducted for one month (about 30 cycles), where one cycle/day was defined as leaving it to stand for 12 hours with the interior of the chamber set at 0° C., followed by leaving it to stand for 12 hours with the interior of the chamber set at 40° C. An appearance of the cosmetic preparation after the test was visually observed, and evaluated in accordance with the following criteria.
A: there were no changes
B: there were changes, but only to the extent that they do not create a problem on product quality
C: there were changes (occurrence of exudation of oil droplets onto the surface, deposition of wax crystals, partial melting of the surface, scratches caused by rubbing against the container and others)
-: no evaluations

### Temporal Stability of Cosmetic Preparation (Hardness)

The cosmetic preparation compositions obtained in the examples and comparative examples were each melted at about 90° C., filled into a predetermined mold for rod-shaped cosmetic preparations (inner diameter 12 mm×length 44 mm, 4.0 g in capacity), and left to stand at −20° C. for 5 minutes to be solidified. The solidified rod-shaped cosmetic preparation was filled into a predetermined container, and left to stand at a room temperature for 6 hours or more. A hardness of the cosmetic preparation composition 6 hours later was considered to be “initial value.” The cosmetic preparation was placed in a cycle testing thermostatic chamber, and a cycle test was conducted for one month (about 30 cycles), where one cycle/day was defined as leaving it to stand for 12 hours with the interior of the chamber set at 0° C., followed by leaving it to stand for 12 hours with the interior of the chamber set at 40° C. After the test, a hardness of the cosmetic preparation composition was measured, and evaluated in accordance with the following criteria.

In this evaluation, a stress (g) when a probe with a diameter of 3 mm was inserted 5 mm into the center of the cosmetic preparation was considered to be the hardness.
A: a hardness reduction from the initial value was less than 30% B: a hardness reduction from the initial value was 30% or more and less than 40%
C: a hardness reduction from the initial value was 40% or more
-: no evaluations

TABLE 1

| | | | | Example | | | | | | | Comparative example | | |
|---|---|---|---|---|---|---|---|---|---|---|---|---|---|
| Component name (all ingredient labeling name) | | | | 1 | 2 | 3 | 4 | 5 | 6 | 7 | 1 | 2 | 3 |
| [symbol] | esahpli | (A) | Hydrogenated castor oil | 8.90 | 8.90 | 8.90 | 8.90 | 8.90 | 8.90 | 8.90 | 8.90 | 8.90 | 8.90 |
| | O | | isostearate | | | | | | | | | | |
| | | | Bis-diglyceryl polyacyladipate-2 | 3.00 | 3.00 | 3.00 | 3.00 | 3.00 | 3.00 | 3.00 | 3.00 | 3.00 | 3.00 |
| | | (B) | PEG-30 dipolyhydroxystearate | 3.00 | 1.50 | 6.00 | 3.00 | — | — | — | — | — | — |
| | | | PEG-20 hydrogenated castor oil | — | — | — | 1.50 | 3.00 | 1.50 | 6.00 | — | — | — |
| | | (B′) | PEG-20 glyceryl triisostearate | — | — | — | — | — | — | — | — | 3.00 | — |
| | | | PEG-100 hydrogenated castor | — | — | — | — | — | — | — | — | — | 3.00 |
| | | | oil | | | | | | | | | | |
| | | (C) | Synthetic wax | 6.70 | 6.70 | 6.70 | 6.70 | 6.70 | 6.70 | 6.70 | 6.70 | 6.70 | 6.70 |
| | | | Beeswax | 2.50 | 2.50 | 2.50 | 2.50 | 2.50 | 2.50 | 2.50 | 2.50 | 2.50 | 2.50 |
| | | | Sunflower seed wax | 0.60 | 0.60 | 0.60 | 0.60 | 0.60 | 0.60 | 0.60 | 0.60 | 0.60 | 0.60 |
| | | (D) | Triethylhexanoin | 29.10 | 30.60 | 26.10 | 27.60 | 29.10 | 30.60 | 26.10 | 31.20 | 30.60 | 30.60 |
| | | | Ethylhexyl palmitate | 10.00 | 10.00 | 10.00 | 10.00 | 10.00 | 10.00 | 10.00 | 10.00 | 10.00 | 10.00 |

| | | | | | | | | | | | |
|---|---|---|---|---|---|---|---|---|---|---|---|
| | Polyglyceryl-10 pentaisostearate | 0.60 | 0.60 | 0.60 | 0.60 | 0.60 | 0.60 | 0.60 | 1.50 | 0.60 | 0.60 |
| | Tocopherol | 0.10 | 0.10 | 0.10 | 0.10 | 0.10 | 0.10 | 0.10 | 0.10 | 0.10 | 0.10 |
| (F) | Spherical cellulose | 15.00 | 15.00 | 15.00 | 15.00 | 15.00 | 15.00 | 15.00 | 15.00 | 15.00 | 15.00 |
| (E) | Water | 20.35 | 20.35 | 20.35 | 20.35 | 20.35 | 20.35 | 20.35 | 20.35 | 20.35 | 20.35 |

| | | | | | | | | | | | |
|---|---|---|---|---|---|---|---|---|---|---|---|
| | Citric acid | 0.02 | 0.02 | 0.02 | 0.02 | 0.02 | 0.02 | 0.02 | 0.02 | 0.02 | 0.02 |
| | Trisodium citrate | 0.13 | 0.13 | 0.13 | 0.13 | 0.13 | 0.13 | 0.13 | 0.13 | 0.13 | 0.13 |
| | Total (mass %) | 100.00 | 100.00 | 100.00 | 100.00 | 100.00 | 100.00 | 100.00 | 100.00 | 100.00 | 100.00 |
| | (A)/(B) (mass ratio) | 3.97 | 7.93 | 1.98 | 2.64 | 3.97 | 7.93 | 1.98 | — | — | — |
| | (B)/((A) + (B) + (C) + (D)) (mass ratio) | 0.047 | 0.024 | 0.094 | 0.071 | 0.047 | 0.024 | 0.094 | — | — | — |
| | (B)/(E) (mass ratio) | 0.15 | 0.07 | 0.29 | 0.22 | 0.15 | 0.07 | 0.29 | 0.0 | 0.0 | 0.0 |
| [symbol] | Moldability | A | A | A | A | A | A | A | A | A | A |
| | Dispersion stability and emulsification | A | B | A | A | A | B | A | C | B | C |
| | stability under remelting | | | | | | | | | | |
| | Usability: light spreadability | B | A | B | B | A | A | B | — | B | — |
| | Usability: moistness | B | A | B | A | A | A | B | — | — | — |
| | Temporal stability: appearance | A | B | A | A | B | B | A | — | C | — |
| | Temporal stability: hardness | B | B | B | B | B | B | B | — | B | — |

[symbol]

indicates data missing or illegible when filed

TABLE 2

| | | | | Example | | | | |
|---|---|---|---|---|---|---|---|---|
| Component name (all ingredient labeling name) | | | | 8 | 9 | 10 | 11 | 12 |
| Water-in-oil | Oil | (A) | Hydrogenated castor oil isostearate | 6.00 | 7.00 | 7.00 | 2.50 | 7.00 |
| type | phase | | Bis-diglyceryl polyacyladipate-2 | 2.50 | 3.00 | 3.00 | 1.50 | 3.00 |
| solid | | (B) | PEG-30 dipolyhydroxystearate | 3.00 | 3.00 | 3.00 | 3.00 | 3.00 |
| emulsified | | | PEG-5 hydrogenated castor oil | — | 3.00 | — | — | — |
| cosmetic | | | PEG-20 hydrogenated castor oil | 1.50 | — | — | 3.00 | 3.00 |
| preparation | | | PEG-60 hydrogenated castor oil | — | — | 3.00 | — | — |
| | | (C) | Synthetic wax | 6.50 | 6.70 | 6.70 | 6.50 | 8.00 |
| | | | Beeswax | 2.00 | 2.50 | 2.50 | 2.00 | 1.50 |
| | | | Sunflower seed wax | 0.50 | 0.60 | 0.60 | 0.50 | 0.50 |
| | | (D) | Triethylhexanoin | 22.30 | 13.50 | 13.50 | 22.30 | 13.30 |
| | | | Ethylhexyl palmitate | — | — | — | — | 10.00 |
| | | | Dodecane | 10.00 | 10.00 | 10.00 | 10.00 | — |

| | | | | | | |
|---|---|---|---|---|---|---|
| | Polyglyceryl-10 pentaisostearate | 0.60 | 0.60 | 0.60 | 0.60 | 0.60 |
| | Tocopherol | 0.10 | 0.10 | 0.10 | 0.10 | 0.10 |
| (F) | Spherical cellulose | 5.00 | — | — | — | 2.50 |
| (E) | Water | 39.85 | 49.70 | 49.70 | 69.70 | 41.20 |

| | | | | | | |
|---|---|---|---|---|---|---|
| | Citric acid | 0.02 | 0.04 | 0.04 | 0.04 | 0.04 |
| | Trisodium citrate | 0.13 | 0.26 | 0.26 | 0.26 | 0.26 |
| | 1,3-butylene glycol | — | — | — | — | 5.00 |
| | Pentylene glycol | — | — | — | — | 1.00 |
| | Total (mass %) | 100.00 | 100.00 | 100.00 | 122.00 | 100.00 |
| | (A)/(B) (mass ratio) | 1.89 | 1.67 | 1.67 | 0.67 | 1.67 |
| | (B)/((A) + (B) + (C) + (D)) (mass ratio) | 0.083 | 0.122 | 0.122 | 0.117 | 0.122 |
| | (B)/(E) (mass ratio) | 0.11 | 0.12 | 0.12 | 0.086 | 0.15 |
| Evaluation | Moldability | A | A | A | A | A |
| | Dispersion stability and emulsification stability | A | A | A | A | A |
| | under remelting | | | | | |
| | Usability: light spreadability | A | A | A | A | B |
| | Usability: moistness | A | A | A | A | B |
| | Temporal stability: appearance | B | B | B | B | B |
| | Temporal stability: hardness | A | A | A | B | B |

### INDUSTRIAL APPLICABILITY

Uses of the water-in-oil type solid emulsified cosmetic preparation of the present invention are not particularly limited, but it can be utilized for, for example, foundations, eye shadows, eye liners, mascaras, concealers, lipsticks, balms and other water-in-oil type solid make-up cosmetic preparations, make-up bases and others.

1. A water-in-oil type solid emulsified cosmetic preparation, comprising the following components, and substantially free of silicone compounds,
(A) a compound which is pasty at 25° C. (hereinafter referred to as “component (A)”),
(B) a compound selected from PEG-30 dipolyhydroxystearate, PEG-5 hydrogenated castor oil, PEG-10 hydrogenated castor oil, PEG-20 hydrogenated castor oil, PEG-30 hydrogenated castor oil, PEG-40 hydrogenated castor oil, PEG-60 hydrogenated castor oil and a combination of two or more of these (hereinafter referred to as “component (B)”),
(C) a solid oil component having a melting point of 65° C. or more (hereinafter referred to as “component (C)”),
(D) a low-viscosity liquid oil component having a viscosity of 40 mPa·s or less at 25° C. (hereinafter referred to as “component (D)”), and
(E) water (hereinafter referred to as “component (E)”).

2. The water-in-oil type solid emulsified cosmetic preparation according to claim 1, wherein the component (B) is a combination of (B1) PEG-30 dipolyhydroxystearate and (B2) any one selected from PEG-5 hydrogenated castor oil, PEG-10 hydrogenated castor oil, PEG-20 hydrogenated castor oil, PEG-30 hydrogenated castor oil, PEG-40 hydrogenated castor oil and PEG-60 hydrogenated castor oil, and a mass ratio of a content of (B1) to a content of (B2), (B1)/(B2), falls within the range of 1 to 5.

3. The water-in-oil type solid emulsified cosmetic preparation according to claim 1, wherein the component (A) is one or two or more selected from hydrogenated castor oil isostearate, dipentaerythrityl hexahydroxystearate, dipentaerythrityl hexa (hydroxystearate/stearate/rosinate), dipentaerythrityl tri-polyhydroxystearate, bis-diglyceryl polyacyladipate-2, phytosteryl macadamiate, (phytosteryl/isostearyl/cetyl/stearyl/behenyl) dimer dilinoleate, di(phytosteryl/octyldodecyl) lauroyl glutamate, di(octyldodecyl/phytosteryl/behenyl) lauroyl glutamate and bis(behenyl/isostearyl/phytosteryl) dimer dilinoleyl dimer dilinoleate.

4. The water-in-oil type solid emulsified cosmetic preparation according to claim 1, wherein the component (C) is one or two or more selected from sunflower seed wax, synthetic waxes, beeswax, candelilla wax, carnauba wax, rice bran wax, microcrystalline waxes, polyethylene, (ethylene/propylene) copolymers, solid paraffin, sumac wax, ozokerite, ceresin and hydrogenated hardened oils.

5. The water-in-oil type solid emulsified cosmetic preparation according to claim 1, wherein the component (D) is one or two or more selected from ethylhexyl palmitate, triethylhexanoin, dodecane, isododecane and isohexadecane.

6. The water-in-oil type solid emulsified cosmetic preparation according to claim 1, wherein a mass ratio of a content of the component (B) to a content of the component (E), (B)/(E), is 0.01 to 0.5.

7. The water-in-oil type solid emulsified cosmetic preparation according to claim 1, wherein the cosmetic preparation is selected from foundations, eye shadows, eye liners, mascaras, concealers, lipsticks, balms and make-up bases.
